(19) United States
(12) Patent Application Publication
(10) Pub. No.: US 2007/0065429 A1
(43) Pub. Date: Mar. 22, 2007

(54) Nogo-receptor antagonists for the treatment of conditions involving amyloid plaques

(75) Inventors: Daniel H.S. Lee, Sudbury, MA (US); Weiwei Li, Staten Island, NY (US)

Correspondence Address: STERNE, KESSLER, GOLDSTEIN & FOX, P.L.L.C., 1100 NEW YORK AVE., N.W., WASHINGTON, DC, 20005, US

(73) Assignees: Biogen Idec MA Inc., Cambridge, MA (US); Yale University, New Haven, CT (US)

(21) Appl. No.: 10/553,669

(22) PCT Filed: Apr. 16, 2004
(86) PCT No.: PCT/US04/11728

§ 371 Date: Aug. 9, 2006

Related U.S. Application Data
(60) Provisional application No. 60/463,424, filed on Apr. 16, 2003.

(51) Int. Cl. A61K 39/395; A61K 38/17

(52) U.S. Cl. 424/143.1; 514/12

(57) ABSTRACT

The invention provides methods for treating diseases involving aberrant amyloid-β (Aβ) peptide deposition, including Alzheimer's Disease, by the administration of Nogo receptor antagonists. The invention also provides method for reducing levels of Aβ peptide in a mammal by the administration of soluble Nogo receptor polypeptides

### FIELD OF THE INVENTION

This invention relates to neurobiology, neurology and pharmacology. More particularly, it relates to methods of treating diseases involving aberrant amyloid-β (Aβ) peptide production and deposition, including Alzheimer's disease, by the administration of Nogo receptor antagonists.

### BACKGROUND OF THE INVENTION

Alzheimer's disease (AD) is a neurodegenerative disorder that results in progressive loss of memory, cognition, reasoning, judgment and emotional stability and ultimately death. A pathologic hallmark of AD is the presence of amyloid plaques in the brain. However, amyloid plaques and vascular amyloid deposits (amyloid angiopathy) also are present in other conditions, for example, in Trisomy 21 (Down's Syndrome), Hereditary Cerebral Hemorrhage with Amyloidosis of the Dutch-Type (HCHWA-D), and Cerebral Amyloid Angiopathy (CAA). The major constituent of amyloid plaques is Aβ peptide, which is derived proteolytically from Amyloid Precursor Protein (APP) by β-secretase (βACE) and γ-secretase (Presenilin-1,2 and associated proteins). APP also is converted to innocuous peptides and protein fragments by α-secretases and γ-secretase. Genetic studies of human familial AD (FAD) have found that mutations in APP and/or Presenilins alter the production of total AB peptide or the ratio of fibrillogenic Aβ42-3 peptide to other APP cleavage products. In addition, mice that express mutant human FAD versions of APP with or without mutant presenilins exhibit amyloid plaque deposition and cognitive impairment.

While Aβ peptides are implicated in AD, there is less certainty regarding which forms of Aβ peptide result in neuronal dysfunction and how they act. The transformation of monomeric Aβ peptide to large amyloid plaque deposits proceeds through several steps and intermediate forms may be causative in the neuronal dysfunction of AD. Accordingly, therapeutic intervention has focused on reducing levels of Aβ peptide and preventing amyloid plaque formation. These approaches have met with some success and include, e.g., immunization with Aβ peptide and passive administration of anti-Aβ-peptide antibodies. See, e.g., Bard et al., Nature Med. 6: 916-19 (2000); Holtzman et al., Adv. Drug Delivery Rev. 54: 1603-13 (2002); and International Patent Application Nos. WO 99/27944, WO 00/72876, and WO 00/72880. However, there remains an urgent need to devise further therapeutic treatments for AD.

### SUMMARY OF THE INVENTION

The present invention is based on the discoveries that treatment with soluble Nogo receptor polypeptides reduces levels of the Aβ peptide and that treatment with a Nogo receptor antagonist, such as a soluble Nogo receptor polypeptide, reduces production of Aβ peptide and plaque deposits. Based on these discoveries, the invention features methods of treating conditions associated with the deposition of amyloid plaques, including Alzheimer's disease, by the administration of soluble fragments of the Nogo receptor polypeptide and Nogo receptor antagonists.

In some embodiments, the invention provides a method for reducing levels of Ab peptide in a mammal, comprising administering a therapeutically effective amount of a soluble Nogo receptor polypeptide. In some embodiments, the levels of Ab peptide are elevated in association with a disease, disorder or condition. In some embodiments, the disease, disorder or condition is Alzheimer's disease.

In some embodiments, the soluble Nogo receptor polypeptide is administered by bolus injection or chronic infusion. In some embodiments, the soluble Nogo receptor polypeptide is administered intravenously. In some embodiments, the soluble Nogo receptor polypeptide is administered directly into the central nervous system. In some embodiments, the soluble Nogo receptor polypeptide is administered directly into a lateral ventricle.

In some embodiments, the soluble Nogo receptor polypeptide is a soluble form of a mammalian NgR1. In some embodiments, the soluble form of a mammalian NgR1: (a) comprises amino acids 26 to 310 of human NgR1 (SEQ ID NO: 3) with up to ten conservative amino acid substitutions; and (b) lacks (i) a functional transmembrane domain, and (ii) a functional signal peptide. In some embodiments, the soluble form of a mammalian NgR1: (a) comprises amino acids 26 to 344 of human NgR1 (SEQ ID NO: 4) with up to ten conservative amino acid substitutions; and (b) lacks (i) a functional transmembrane domain, and (ii) a functional signal peptide. In some embodiments, the soluble form of a mammalian NgR1: (a) comprises amino acids 27 to 310 of rat NgR1 (SEQ ID NO: 5) with up to ten conservative amino acid substitutions; and (b) lacks (i) a functional transmembrane domain, and (ii) a functional signal peptide. In some embodiments, the soluble form of a mammalian NgR1: (a) comprises amino acids 27 to 344 of rat NgR1 (SEQ ID NO: 6) with up to ten conservative amino acid substitutions; and (b) lacks (i) a functional transmembrane domain, and (ii) a functional signal peptide.

In some embodiments, the soluble form of a mammalian NgR1 further comprises a fusion moiety. In some embodiments, the fusion moiety is an immunoglobulin moiety. In some embodiments, the immunoglobulin moiety is an Fc moiety.

In some embodiments, the therapeutically effective amount is from 0.001 mg/kg to 10 mg/kg. In some embodiments, the therapeutically effective amount is from 0.01 mg/kg to 1.0 mg/kg. In some embodiments, the therapeutically effective amount is from 0.05 mg/kg to 0.5 mg/kg.

In some embodiments, the invention provides a method of preventing or treating a disease, disorder or condition associated with plaques of Ab peptide in a mammal, comprising administering a therapeutically effective amount of an NgR1 antagonist. In some embodiments, the plaques are in the central nervous system. In some embodiments, the disease, disorder or condition is Alzheimer's Disease.

In some embodiments, the NgR1 antagonist is administered directly into the central nervous system. In some embodiments, the NgR1 antagonist is administered directly into the a lateral ventricle. In some embodiments, the NgR1 antagonist is administered by bolus injection or chronic infusion.

In some embodiments, the soluble Nogo receptor polypeptide is a soluble form of a mammalian NgR1. In some embodiments, the soluble form of a mammalian NgR1: (a) comprises amino acids 26 to 310 of human NgR1 (SEQ ID NO: 3) with up to ten conservative amino acid substitutions; and (b) lacks (i) a functional transmembrane domain, and (ii) a functional signal peptide. In some embodiments, the soluble form of a mammalian NgR1: (a) comprises amino acids 26 to 344 of human NgR1 (SEQ ID NO: 4) with up to ten conservative amino acid substitutions; and (b) lacks (i) a functional transmembrane domain, and (ii) a functional signal peptide. In some embodiments, the soluble form of a mammalian NgR1: (a) comprises amino acids 27 to 310 of rat NgR1 (SEQ ID NO: 5) with up to ten conservative amino acid substitutions; and (b) lacks (i) a functional transmembrane domain, and (ii) a functional signal peptide. In some embodiments, the soluble form of a mammalian NgR1: (a) comprises amino acids 27 to 344 of rat NgR1 (SEQ ID NO: 6) with up to ten conservative amino acid substitutions; and (b) lacks (i) a functional transmembrane domain, and (ii) a functional signal peptide.

In some embodiments, the soluble form of a mammalian NgR1 further comprises a fusion moiety. In some embodiments, the fusion moiety is an immunoglobulin moiety. In some embodiments, the immunoglobulin moiety is an Fc moiety.

In some embodiments, the NgR1 antagonist comprises an antibody or antigen-binding fragment thereof that binds to a mammalian NgR1. In some embodiments, the antibody is selected from the group consisting of a polyclonal antibody, a monoclonal antibody, a Fab fragment, a Fab′ fragment, a F(ab′)2 fragment, an Fv fragment, an Fd fragment, a diabody, and a single-chain antibody. In some embodiments, the antibody or antigen-binding fragment thereof binds to an polypeptide bound by a monoclonal antibody produced by a hybridoma selected from the group consisting of: HB 7E11 (ATCC® accession No. PTA-4587), HB 1H2 (ATCC® accession No. PTA-4584), HB 3G5 (ATCC® accession No. PTA-4586), HB 5B10 (ATCC® accession No. PTA-4588) and HB 2F7 (ATCC® accession No. PTA-4585). In some embodiments, the monoclonal antibody is produced by the HB 7E11 hybridoma. In some embodiments, the polypeptide comprises an amino acid sequence selected from the group consisting of:

| | |
|---|---|
| AAAFGLTLLEQLDLSDNAQLR; | (SEQ ID NO: 7) |
| LDLSDNAQLR; | (SEQ ID NO: 8) |
| LDLSDDAELR; | (SEQ ID NO: 9) |
| LDLASDNAQLR; | (SEQ ID NO: 10) |
| LDLASDDAELR; | (SEQ ID NO: 11) |
| LDALSDNAQLR; | (SEQ ID NO: 12) |
| LDALSDDAELR; | (SEQ ID NO: 13) |
| LDLSSDNAQLR; | (SEQ ID NO: 14) |
| LDLSSDEAELR; | (SEQ ID NO: 15) |
| DNAQLRVVDPTT; | (SEQ ID NO: 16) |
| DNAQLR; | (SEQ ID NO: 17) |
| ADLSDNAQLRVVDPTT; | (SEQ ID NO: 18) |
| LALSDNAQLRVVDPTT; | (SEQ ID NO: 19) |
| LDLSDNAALRVVDPTT; | (SEQ ID NO: 20) |
| LDLSDNAQLHVVDPTT; | (SEQ ID NO: 21) |
| and | |
| LDLSDNAQLAVVDPTT. | (SEQ ID NO: 22) |

In some embodiments, the therapeutically effective amount is from 0.001 mg/kg to 10 mg/kg. In some embodiments, the therapeutically effective amount is from 0.01 mg/kg to 1.0 mg/kg. In some embodiments, the therapeutically effective amount is from 0.05 mg/kg to 0.5 mg/kg.

### DETAILED DESCRIPTION OF THE INVENTION

Unless defined otherwise, all technical and scientific terms used herein have the same meaning as commonly understood by one of ordinary skill in the art to which this invention belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present invention, suitable methods and materials are described below. The materials, methods and examples are illustrative only and are not intended to be limiting. Other features and advantages of the invention will be apparent from the detailed description and from the claims.

Throughout this specification and claims, the word “comprise,” or variations such as “comprises” or “comprising,” indicate the inclusion of any recited integer or group of integers but not the exclusion of any other integer or group of integers.

In order to further define this invention, the following terms and definitions are provided.

As used herein, “antibody” means an intact immunoglobulin, or an antigen-binding fragment thereof. Antibodies of this invention can be of any isotype or class (e.g., M, D, G, E and A) or any subclass (e.g., G1-4, A1-2) and can have either a kappa (κ) or lambda (λ) light chain.

As used herein, “humanized antibody” means an antibody in which at least a portion of the non-human sequences are replaced with human sequences. Examples of how to make humanized antibodies may be found in U.S. Pat. Nos. 6,054,297, 5,886,152 and 5,877,293.

As used herein, a “therapeutically effective amount” refers to an amount effective, at dosages and for periods of time necessary, to achieve the desired therapeutic result.

As used herein, a “prophylactically effective amount” refers to an amount effective, at dosages and for periods of time necessary to achieve the desired prophylactic result. Typically, since a prophylactic dose is used in subjects prior to or at an earlier stage of disease, the prophylactically effective amount will be less than the therapeutically effective amount.

As used herein, a “patient” means a mammal, e.g., a human.

As used herein, “fusion protein” means a protein comprising a first polypeptide fused to a second, heterologous, polypeptide.

As used herein, a “Nogo receptor antagonist” means a molecule that inhibits the binding of Nogo receptor-1 to a ligand (e.g., NogoA, NogoB, NogoC, MAG, OM-gp).

As used herein, “Nogo receptor polypeptide” includes both full-length Nogo receptor-1 protein and fragments thereof that bind Aβ peptide or antagonize Nogo receptor function.

A first aspect of the invention is based on the discovery that soluble Nogo receptor polypeptides bind directly to Aβ peptide. Therefore, without intending to be bound by theory, it appears that soluble Nogo receptor polypeptides can function as an Aβ peptide sink in vivo. This mechanism can be exploited to deplete Aβ peptide levels in circulating blood, at the site of deposition, or both, thereby inhibiting amyloid plaque formation or reducing the size of existing plaques. Because one site of action is in the bloodstream, the invention advantageously avoids a requirement to administer the soluble Nogo receptor polypeptides to the central nervous system (CNS). It will be appreciated, however, that soluble Nogo receptor polypeptides can be administered directly into the CNS instead of, or in addition to, systemic administration.

A second aspect of the invention is based on the discovery that soluble Nogo receptor polypeptides or other Nogo receptor antagonists, e.g. an anti-Nogo-receptor antibody, interfere with Nogo receptor function in the CNS. This results in both reduced Aβ peptide levels and a reduction in plaque deposits. In this mechanism, the site of action of the soluble Nogo receptor polypeptides or other Nogo receptor antagonists is in the CNS. Without intending to be bound by theory, it appears that at least one effect of inhibiting NgR function is to reduce the processing of APP that yields the Aβ peptide.

Nogo Receptor Antagonists

Any Nogo receptor antagonist may be used in the methods of the invention. For example, Nogo receptor antagonists that may be used in the methods of the invention include, but are not limited to: soluble Nogo receptor-1 polypeptides; antibodies that bind to the Nogo receptor protein and antigen-binding fragments of such antibodies; and small molecule antagonists.

Soluble Nogo Receptor-1 Polypeptides

Some embodiments of the invention use a soluble Nogo receptor-1 polypeptide (Nogo receptor-1 is also variously referred to as “Nogo receptor,” “NogoR,” “NogoR-1,” “NgR,” and “NgR-1”). Full-length Nogo receptor-1 consists of a signal sequence, a N-terminus region (NT), eight leucine-rich repeats (LRR), a LRRCT region (a leucine-rich-repeat domain C-terminal of the eight leucine-rich repeats), a C-terminus region (CT) and a GPI anchor. The sequences of human and rat Nogo receptor polypeptides are shown in Table 1.

TABLE 1

Sequences of Human and Rat Nogo receptor-1
Polypeptides

| | |
|---|---|
| Human | MKRASAGGSRLLAWVLWLQAWQVAAPCPGACVCYNEP |
| Nogo receptor | KVTTSCPQQGLQAVPVGIPAASQRIFLHGNRISHVPA |
| Polypeptide | ASFRACRNLTILWLHSNVLARIDAAAFTGLALLEQLD |
| SEQ ID NO: 1 | LSDNAQLRSVDPATFHGLGRLHTLHLDRCGLQELGPG |
| | LFRGLAALQYLYLQDNALQALPDDTFRDLGNLTHLFL |
| | HGNRISSVPERAFRGLHSLDRLLLHQNRVAHVHPHAF |
| | RDLGRLMTLYLFANNLSALPTEALAPLRALQYLRLND |
| | NPWVCDCRARPLWAWLQKFRGSSSEVPCSLPQRLAGR |
| | DLKRLAANDLQGCAVATGPYHPIWTGRATDEEPLGLP |
| | KCCQPDAADKA |
| Rat | MKRASSGGSRLPTWVLWLQAWRVATPCPGACVCYNEP |
| Nogo receptor | KVTTSRPQQGLQAVPAGIPASSQRIFLHGNRISYVPA |
| Polypeptide | ASFQSCRNLTILWLHSNALAGIDAAAAFTGLTLLEQL |
| SEQ ID NO: 2 | DLSDNAQLRVVDPTTFRGLGHLHTLHLDRCGLQELGP |
| | GLFRGLAALQYLYLQDNNLQALPDNTFRDLGNLTHLF |
| | LHGNRIPSVPEHAFRGLHSLDRLLLHQNHVARVHPHA |
| | FRDLGRLMTLYLFANNLSMLPAEVLVPLRSLQYLRLN |
| | DNPWVCDCRARPLWAWLQKFRGSSSGVPSNLPQRLAG |
| | RDLKRLATSDLEGCAVASGPFRPFQTNQLTDEELLGL |
| | PKCCQPDAADKA |

Soluble Nogo receptor polypeptides used in the methods of the invention comprise an NT domain; 8 LRRs and an LRRCT domain and lack a signal sequence and a functional GPI anchor (i.e., no GPI anchor or a GPI anchor that fails to efficiently associate to a cell membrane). Suitable polypeptides include, for example, amino acids 26-310 (SEQ ID NO: 3) and 26-344 (SEQ ID NO: 4) of the human Nogo receptor and amino acids 27-310 (SEQ ID NO: 5) and 27-344 (SEQ ID NO: 6) of the rat Nogo receptor (Table 2). Additional polypeptides which may be used in the methods of the invention are described, for example, in International Patent Applications PCT/US02/32007 and PCT/US03/25004.

TABLE 2

Soluble Nogo receptor Polypeptides
from Human and Rat

| | |
|---|---|
| Human 26-310 | PCPGACVCYNEPKVTTSCPQQGLQAVPVGIPAASQRI |
| SEQ ID NO: 3 | FLHGNRISHVPAASFRACRNLTILWLHSNVLARIDAA |
| | AFTGLALLEQLDLSDNAQLRSVDPATFHGLGRLHTLH |
| | LDRCGLQELGPGLFRGLAALQYLYLQDNALQALPDDT |
| | FRDLGNLTHLFLHGNRISSVPERAFRGLHSLDRLLLH |
| | QNRVAHVHPHAFRDLGRLMTLYLFANNLSALPTEALA |
| | PLRALQYLRLNDNPWVCDCRARPLWAWLQKFRGSSSE |
| | VPCSLPQRLAGRDLKRLAANDLQGCA |
| Human 26-344 | PCPGACVCYNEPKVTTSCPQQGLQAVPVGIPAASQRI |
| SEQ ID NO: 4 | FLHGNRISHVPAASFRACRNLTILWLHSNVLARIDAA |
| | AFTGLALLEQLDLSDNAQLRSVDPATFHGLGRLHTLH |
| | LDRCGLQELGPGLFRGLAALQYLYLQDNALQALPDDT |
| | FRDLGNLTHLFLHGNRISSVPERAFRGLHSLDRLLLH |
| | QNRVAHVHPHAFRDLGRLMTLYLFANNLSALPTEALA |
| | PLRALQYLRLNDNPWVCDCRARPLWAWLQKFRGSSSE |
| | VPCSLPQRLAGRDLKRLAANDLQGCAVATGPYHPIWT |
| | GRATDEEPLGLPKCCQPDAADKA |
| Rat 27-310 | CPGACVCYNEPKVTTSRPQQGLQAVPAGIPASSQRIF |
| SEQ ID NO: 5 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCA |
| Rat 27-344 | CPGACVCYNEPKVTTSRPQQGLQAVPAGIPASSQRIF |
| SEQ ID NO: 6 | LHGNRISYVPAASFQSCRNLTILWLHSNALAGIDAAA |
| | FTGLTLLEQLDLSDNAQLRVVDPTTFRGLGHLHTLHL |
| | DRCGLQELGPGLFRGLAALQYLYLQDNNLQALPDNTF |
| | RDLGNLTHLFLHGNRIPSVPEHAFRGLHSLDRLLLHQ |
| | NHVARVHPHAFRDLGRLMTLYLFANNLSMLPAEVLVP |
| | LRSLQYLRLNDNPWVCDCRARPLWAWLQKFRGSSSGV |
| | PSNLPQRLAGRDLKRLATSDLEGCAVASGPFRPFQTN |
| | QLTDEELLGLPKCCQPDAADKA |

A fusion protein that includes a soluble Nogo receptor polypeptide may be used in the methods of the invention. In some embodiments, the heterologous moiety of the fusion protein is an immunoglobulin constant domain. In some embodiments, the immunoglobulin constant domain is a heavy chain constant domain. In some embodiments, the heterologous polypeptide is an Fc fragment. In some embodiments, the Fc is joined to the C-terminal end of a soluble Nogo receptor polypeptide. In some embodiments, the fusion Nogo receptor protein is a dimer, e.g., an Fc fusion dimer.

Antibodies

Some methods of the invention use a Nogo receptor antagonist that is an antibody or an antigen-binding fragment thereof that specifically binds an immunogenic Nogo receptor-1 polypeptide and inhibits the binding of Nogo receptor-1 to a ligand (e.g., NogoA, NogoB, NogoC, MAG, OM-gp). The antibody or antigen-binding fragment used in these methods of the invention may be produced in vivo or in vitro. In some embodiments, the anti-Nogo receptor-1 antibody or antigen-binding fragment thereof is murine or human. In some embodiments, the anti-Nogo receptor-1 antibody or antigen-binding fragment thereof is recombinant, engineered, humanized and/or chimeric. In some embodiments, the antibody is selected from the antibodies described in International Patent Application No. PCT/US03/25004. Antibodies useful in the present invention may be employed with or without modification.

Exemplary antigen-binding fragments of the antibodies which may be used in the methods of the invention are Fab, Fab′, F(ab′)₂, Fv, Pd, dAb, and fragments containing complementarity determining region (CDR) fragments, single-chain antibodies (scFv), chimeric antibodies, diabodies and polypeptides that contain at least a portion of an immunoglobulin that is sufficient to confer specific antigen-binding to the polypeptide (e.g., immunoadhesins).

As used herein, Fd means a fragment that consists of the V_{H} and C_{H1} domains; Fv means a fragment that consists of the V_{L} and V_{H} domains of a single arm of an antibody; and dAb means a fragment that consists of a V_{H} domain (Ward et al., Nature 341:544-46 (1989)). As used herein, single-chain antibody (scFv) means an antibody in which a V_{L} region and a V_{H} region are paired to form a monovalent molecules via a synthetic linker that enables them to be made as a single protein chain (Bird et al., Science 242:423-26 (1988) and Huston et al., Proc. Natl. Acad. Sci. USA 85:5879-83 (1988)). As used herein, diabody means a bispecific antibody in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites (see, e.g., Holliger et al., Proc. Natl. Acad. Sci. USA 90:6444-48 (1993) and Poljak et al., Structure 2:1121-23 (1994)).

Immunization

Antibodies for use in the methods of the invention can be generated by immunization of a suitable host (e.g., vertebrates, including humans, mice, rats, sheep, goats, pigs, cattle, horses, reptiles, fishes, amphibians, and in eggs of birds, reptiles and fish). Such antibodies may be polyclonal or monoclonal. For a review of methods for making antibodies see, e.g., Harlow and Lane (1988), Antibodies, A Laboratory Manual; Yelton et al., Ann. Rev. of Biochem., 50:657-80 (1981); and Ausubel et al. (1989), Current Protocols in Molecular Biology (New York: John Wiley & Sons). Immunoreactivity of an antibody with an immunogenic Nogo receptor polypeptide may be determined by any suitable method, including, e.g., immunoblot assay and ELISA. Monoclonal antibodies for use in the methods of the invention can be made by conventional procedures as described, e.g., in Harlow and Lane (1988), supra.

A host may be immunized with an immunogenic Nogo receptor-1 polypeptide, either with or without an adjuvant. Suitable polypeptides are described in, for example, International Patent Applications PCT/US01/31488, PCT/US02/32007 and PCT/US03/25004. The host also may be immunized with Nogo receptor-1 associated with the cell membrane of an intact or disrupted cell and antibodies identified by binding to a Nogo receptor-1 polypeptide. Other suitable techniques for producing an antibody involve in vitro exposure of lymphocytes to the Nogo receptor-1 or to an immunogenic polypeptide of the invention, or alternatively, selection of libraries of antibodies in phage or similar vectors. See Huse et al., Science 246:1275-81 (1989).

Anti-Nogo receptor-1 antibodies used in the methods of this invention also can be isolated by screening a recombinant combinatorial antibody library. Methodologies for preparing and screening such libraries are known in the art. There are commercially available methods and materials for generating phage display libraries (e.g., the Pharmacia Recombinant Phage Antibody System, catalog no. 27-9400-01; the Stratagene SurfZAP™ phage display kit, catalog no. 240612; and others from MorphoSys). Following screening and isolation of an anti-Nogo receptor-1 antibody from a recombinant immunoglobulin display library, the nucleic acid encoding the selected antibody can be recovered from the display package (e.g., from the phage genome) and subcloned into other expression vectors by standard recombinant DNA techniques. To express an antibody isolated by screening a combinatorial library, DNA encoding the antibody heavy chain and light chain or the variable regions thereof is cloned into a recombinant expression vector and introduced into a host cell.

Uses for Nogo Receptor Antagonists

This invention relates to methods for treating diseases involving aberrant Aβ peptide deposition by administering Nogo receptor antagonists. Nogo receptor antagonists used in the methods of the invention include, but are not limited to, soluble Nogo receptor polypeptides, antibodies to the Nogo receptor protein and antigen-binding fragments thereof, and small molecule antagonists. In some embodiments, the aberrant Aβ peptide deposition is associated with a disease, disorder or condition, e.g., Alzheimer's disease.

Uses for Soluble Nogo Receptor Polypeptides

This invention also relates to methods for reducing levels of Aβ peptide by the administration of soluble Nogo receptor polypeptides. In some of these embodiments, the levels of Aβ peptide are elevated in association with a disease, disorder or condition, e.g., Alzheimer's disease.

Pharmaceutical Compositions

The soluble Nogo receptor polypeptides and Nogo receptor antagonists used in the methods of the invention may be formulated into pharmaceutical compositions for administration to mammals, including humans. The pharmaceutical compositions used in the methods of this invention comprise pharmaceutically acceptable carriers.

Pharmaceutically acceptable carriers useful in these pharmaceutical compositions include, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions used in the methods of the present invention may be administered by any suitable method, e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. As described previously, Nogo receptor antagonists used in the methods of the invention act in the CNS, which results in both reduced Aβ peptide levels and a reduction in plaque deposits. Accordingly, in methods of the invention that use a Nogo receptor antagonist, the Nogo receptor antagonist must cross the blood-brain barrier. This crossing can result from the physico-chemical properties inherent in the Nogo receptor antagonist molecule itself, from other components in a pharmaceutical formulation, or from the use of a mechanical device such as a needle, cannula or surgical instruments to breach the blood-brain barrier. Where the Nogo receptor antagonist is a molecule that does not inherently cross the blood-brain barrier, suitable routes of administration are, e.g., intrathecal or intracranial, e.g., directly into a lateral ventricle. Where the Nogo receptor antagonist is a molecule that inherently crosses the blood-brain barrier—or where a soluble Nogo receptor polypeptide is used in a method of the invention where direct binding to Aβ peptide results in reduced Aβ peptide levels—the route of administration may be by one or more of the various routes described below.

Sterile injectable forms of the compositions used in the methods of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Parenteral formulations may be a single bolus dose, an infusion or a loading bolus dose followed with a maintenance dose. These compositions may be administered once a day or on an “as needed” basis.

Certain pharmaceutical compositions used in the methods of this invention may be orally administered in any orally acceptable dosage form including, e.g., capsules, tablets, aqueous suspensions or solutions. Certain pharmaceutical compositions also may be administered by nasal aerosol or inhalation. Such compositions may be prepared as solutions in saline, employing benzyl alcohol or other suitable preservatives, absorption promoters to enhance bioavailability, fluorocarbons, and/or other conventional solubilizing or dispersing agents.

The amount of a soluble Nogo receptor polypeptide or a Nogo receptor antagonist that may be combined with the carrier materials to produce a single dosage form will vary depending upon the host treated and the particular mode of administration. The composition may be administered as a single dose, multiple doses or over an established period of time in an infusion. Dosage regimens also may be adjusted to provide the optimum desired response (e.g., a therapeutic or prophylactic response).

The methods of the invention use a “therapeutically effective amount” or a “prophylactically effective amount” of a soluble Nogo receptor polypeptide or a Nogo receptor antagonist. Such a therapeutically or prophylactically effective amount may vary according to factors such as the disease state, age, sex, and weight of the individual. A therapeutically or prophylactically effective amount is also one in which any toxic or detrimental effects are outweighed by the therapeutically beneficial effects.

A specific dosage and treatment regimen for any particular patient will depend upon a variety of factors, including the particular soluble Nogo receptor polypeptide or Nogo receptor antagonist used, the patient's age, body weight, general health, sex, and diet, and the time of administration, rate of excretion, drug combination, and the severity of the particular disease being treated. Judgment of such factors by medical caregivers is within ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well-known in the art.

In the methods of the invention the Nogo receptor antagonists are generally administered directly to the CNS, intracerebroventricularly, or intrathecally, e.g. into a lateral ventricle. In methods of the invention where a soluble Nogo receptor polypeptide is used for reducing levels of Aβ peptide, the soluble Nogo receptor polypeptides are generally administered intravenously. Compositions for administration according to the methods of the invention can be formulated so that a dosage of 0.001-10 mg/kg body weight per day of the Nogo receptor antagonist is administered. In some embodiments of the invention, the dosage is 0.01-1.0 mg/kg body weight per day. In some embodiments, the dosage is 0.05-0.5 mg/kg body weight per day.

Supplementary active compounds also can be incorporated into the compositions used in the methods of the invention. For example, a Nogo receptor antibody or an antigen-binding fragment thereof, or a soluble Nogo receptor polypeptide or a fusion protein may be coformulated with and/or coadministered with one or more additional therapeutic agents.

The invention encompasses any suitable delivery method for a soluble Nogo receptor polypeptide or a Nogo receptor antagonist to a selected target tissue, including bolus injection of an aqueous solution or implantation of a controlled-release system. Use of a controlled-release implant reduces the need for repeat injections.

The soluble Nogo receptor polypeptide or Nogo receptor antagonists used in the methods of the invention may be directly infused into the brain. Various implants for direct brain infusion of compounds are known and are effective in the delivery of therapeutic compounds to human patients suffering from neurological disorders. These include chronic infusion into the brain using a pump, stereotactically implanted, temporary interstitial catheters, permanent intracranial catheter implants, and surgically implanted biodegradable implants. See, e.g., Gill et al., supra; Scharfen et al., “High Activity Iodine-125 Interstitial Implant For Gliomas,” Int. J. Radiation Oncology Biol. Phys. 24(4):583-91 (1992); Gaspar et al., “Permanent ¹²⁵I Implants for Recurrent Malignant Gliomas,” Int. J. Radiation Oncology Biol. Phys. 43(5):977-82 (1999); chapter 66, pages 577-580, Bellezza et al., “Stereotactic Interstitial Brachytherapy,” in Gildenberg et al., Textbook of Stereotactic and Functional Neurosurgery, McGraw-Hill (1998); and Brem et al., “The Safety of Interstitial Chemotherapy with BCNU-Loaded Polymer Followed by Radiation Therapy in the Treatment of Newly Diagnosed Malignant Gliomas: Phase I Trial,” J. Neuro-Oncology 26:111-23 (1995).

The compositions may also comprise a soluble Nogo receptor polypeptide or a Nogo receptor antagonist dispersed in a biocompatible carrier material that functions as a suitable delivery or support system for the compounds. Suitable examples of sustained release carriers include semipermeable polymer matrices in the form of shaped articles such as suppositories or capsules. Implantable or microcapsular sustained release matrices include polylactides (U.S. Pat. No. 3,773,319; EP 58,481), copolymers of L-glutamic acid and gamma-ethyl-L-glutamate (Sidman et al., Biopolymers 22:547-56 (1985)); poly(2-hydroxyethyl-methacrylate), ethylene vinyl acetate (Langer et al., J. Biomed. Mater. Res. 15:167-277 (1981); Langer, Chem. Tech. 12:98-105 (1982)) orpoly-D-(−)-3hydroxybutyric acid (EP 133,988).

In some embodiments of the invention, a soluble Nogo receptor polypeptide or Nogo receptor antagonist is administered to a patient by direct infusion into an appropriate region of the brain. See, e.g., Gill et al., “Direct brain infusion of glial cell line-derived neurotrophic factor in Parkinson disease,” Nature Med. 9: 589-95 (2003). Alternative techniques are available and may be applied to administer a soluble Nogo receptor polypeptide or Nogo receptor antagonist according to the invention. For example, stereotactic placement of a catheter or implant can be accomplished using the Riechert-Mundinger unit and the ZD (Zamorano-Dujovny) multipurpose localizing unit. A contrast-enhanced computerized tomography (CT) scan, injecting 120 ml of omnipaque, 350 mg iodine/ml, with 2 mm slice thickness can allow three-dimensional multiplanar treatment planning (STP, Fischer, Freiburg, Germany). This equipment permits planning on the basis of magnetic resonance imaging studies, merging the CT and MRI target information for clear target confirmation.

The Leksell stereotactic system (Downs Surgical, Inc., Decatur, Ga.) modified for use with a GE CT scanner (General Electric Company, Milwaukee, Wis.) as well as the Brown-Roberts-Wells (13RW) stereotactic system (Radionics, Burlington, Mass.) can be used for this purpose. Thus, on the morning of the implant, the annular base ring of the BRW stereotactic frame can be attached to the patient's skull. Serial CT sections can be obtained at 3 mm intervals though the (target tissue) region with a graphite rod localizer frame clamped to the base plate. A computerized treatment planning program can be run on a VAX 11/780 computer (Digital Equipment Corporation, Maynard, Mass.) using CT coordinates of the graphite rod images to map between CT space and BRW space.

### EXAMPLES

### Example 1

### Subcellular Localization of NgR and Nogo is Altered in Alzheimer's Disease

We obtained anonymous human Alzheimer's Disease (AD) and control brain tissue samples from the NIH-supported Harvard Brain Tissue Resource Center and examined them histologically for NogoA and NgR localization using anti-NogoA and anti-NgR antibodies (see Wang et al., J. Neurosci. 22: 5505-15 (2002)). Tissue from the hippocampus and Broadman's area 44 were examined in six control and six AD cases. Specificity of staining was confirmed by antigen blockade and by the presence of a single immunoreactive band on immunoblots.

In the control adult human brain, NogoA immunoreactivity was detectable in a diffuse granular pattern in the neuropil of these brain regions with little cellular staining. In contrast, in all of the AD cases, there was a dramatic shift of NogoA to neuronal cell bodies. NgR localization was shifted in an opposite fashion. In control cases, the highest concentration of the NgR protein was found in cell soma, whereas in the AD cases the brain exhibited a diffuse neuropil immunoreactivity and little cellular staining. Immunoblot analysis with anti-NgR antibodies confirmed that this was not due to altered levels of NgR and adjacent staining with anti-NogoA antibodies clearly indicated that this was not due to an absence of neurons. In addition to the shift of NgR out of the cell soma, we observed that NgR was concentrated in amyloid plaques and double immunohistochemistry for Aβ and NgR demonstrated that the two proteins co-localize in these deposits. These findings suggested that the NogoA/NgR pathway has a role in AD pathology.

### Example 2

### APP and Multiple Forms of Aβ Peptide Interact with NgR

Based on these observations, we tested whether NogoA or NgR interacts directly with APP. Epitope-tagged constructs of NgR (NgR-myc constructed as described in Liu et al., Science 297: 1190-93 (2002)) and APP (APP-V5; I.M.A.G.E. clone #5259793 was subcloned into pcDNA3.1-V5His to create C-terminal fusion to APP-695) were expressed in COS-7 cells and immunoprecipitation with anti-V5 and anti-myc antibodies was performed. Immunoblots of the immunoprecipitated material were then probed with anti-V5, anti-myc and anti-NgR antibodies. The immunoprecipitation studies demonstrated a specific association of APP with NgR. NogoA was not detectable in the imunoprecipitated material using anti-NogoA. We also monitored the location of epitope-tagged APP in the transfected COS-7 cells and found that a majority of the protein in controls was localized intracellularly in perinuclear regions, but that co-expression of NgR with APP shifts a majority of APP to the cell surface. In addition, APP and NgR localizations were identical in double-labeling experiments in the transfected cells. The total level of cellular APP expression was not altered by NgR co-expression. Furthermore, the native APP and NgR proteins also were co-localized in primary neurons as determined by probing with anti-APP (Santa Cruz Biotechnology) and anti-NgR antibodies. These results confirm a physical association of NgR with APP.

We then investigated whether the Aβ region of APP is involved in its interaction with NgR—including whether the fibrillogenic Aβ42-3 peptide binds NgR. We created two fusion protein constructs containing alkaline phosphatase (AP) and the hydrophilic region of Aβ (amino acids 1-28) by fusing the coding sequence in frame with the signal sequence-6×His-placental alkaline phosphatase (AP) sequence of the vector pAP-6 (Nakamura et al., Neuron 2: 1093-1100, 1988). The AP-Aβ and Aβ-AP proteins both bound to NgR-expressing COS cells but not to vector-transfected COS cells. This binding was saturable with an apparent K_{d} of 60 nM. The interaction also was detected using purified Biotin-Aβ(1-40) in an ELISA-type assay with immobilized NgR. In contrast, the reverse 40-1 peptide did not interact with immobilized NgR in any of these experiments. We also incubated Fluo-Aβ42 for 2 h at 4° C. with human SKNMC cells expressing human NgR-1 and found that the fibrillogenic Aβ42 peptide binds to these cells.

We also tested the binding of Aβ peptide to a soluble NgR polypeptide, sNgR310 (see, e.g., PCT/US03/25004), as follows. sNgR310 was immobilized on a microtiter plate and Biotin-Aβ1-40 or Biotin-Aβ40-1 was applied for 16 h at 4° C. After removing unbound peptides, bound Biotin-Aβ was detected by streptavidin conjugated HRP. As with full-length NgR we observed that Biotin-Aβ1-40, but not Biotin-Aβ40-1 bound to sNgR310. We also performed these experiments in the presence of anti-NgR antibodies, such as monoclonal antibody HB 7E11 (described in PCT/US03/25004), and found that binding of Biotin-Aβ1-40 can be inhibited by the anti-NgR antibodies. In separate experiments, we confirmed that anti-NgR anitobides also inhibit binding of Biotin-Aβ1-40 to either COS7 cells expression rat NgR1 or to SKNMC cells expressing human NgR1. Collectively, these data confirmed that APP and the naturally occurring forms of Aβ peptide interact directly with NgR.

The specificity and selectivity of the Aβ(1-28) interaction with NgR was probed in several ways. The interaction was specific for NgR1 because neither NgR2 or NgR3—which share sequence similarity with NgR—bound Aβ-AP. Further, we observed species specificity: human NgR binds human AB to a greater extent than mouse NgR binds human AB or human NgR binds mouse AB or mouse NgR binds mouse Aβ. Finally, we examined neurons cultured from ngr −/− mice generated in our laboratory (these mice are deleted for Exon II of NgR and no NgR protein is produced) and found that they do not bind either the Nogo-66 fragment of NogoA (see, e.g. International Patent Applications PCT/US01/01041 and PCT/US02/32007) or the Aβ peptide. These data demonstrated that NgR is the primary neuronal-cell-surface binding site for Aβ(1-28).

To further define which residues are required for NgR interaction, we created AP fusions of several deletions in the Aβ(1-28) peptide and monitored binding to NgR expressed in COS-7 cells by assaying AP activity. Deletion of the amino-terminal 7 residues did not alter binding to NgR and deletion of the amino-terminal 14 residues moderately reduced NgR binding. However, deletion of amino acids 1-16 abrogated NgR binding. At the carboxy terminus of Aβ (1-28), a seven-amino-acid-truncation mutant exhibited no affinity for NgR. Thus, amino acids 7-28 are involved in NgR affinity and amino acids 15-28 are especially important. Consistent with these observations, we found that the native β-secretase peptide products (containing amino acids 8-21) but not α-secretase cleavage products (proteolyzed at amino acid 17) bound NgR.

### Example 3

### Aβ Binds a NgR Site Distinct from that Bound by the Myelin Ligands

We analyzed whether Aβ (1-28) binding competed for NgR binding with other known ligands for NgR by allowing 250 nM soluble AP-Aβ(1-28) or AP-Nogo (1-33) to bind to wells coated with purified sNgR310-Fc in the presence of various concentrations of competing free Aβ. In a similar experiment, we also tested whether Biotin-Aβ(1-40) binds to rat sNgP344-Fc. We observed that the Aβ peptides bind to both sNgR310-Fc and sNgR344-Fc. Thus, Aβ peptides—like other ligands of NgR—requires the entire LRR region of the NgR protein for binding, but does not require the carboxyl tail from residues 310-450. However, Aβ(1-28) displaced Aβ-AP binding but not AP-Nogo-66 (1-33) or AP-OMgp binding in competition assays. The Aβ peptide may begin to displace AP-MAG very slightly at high concentrations in our experiments. Thus, the Aβ binding site on NgR appears largely distinct from that for the myelin ligands NogoA, OMgp and MAG. Consistent with this, the presence of Aβ had little effect on myelin or Nogo-66 inhibition of neurite outgrowth.

### Example 4

### NgR Enhances Aβ Production

Because one of the critical steps in the development of AD is the proteolytic production of Aβ from APP, we assessed the effect of NgR on this processing. We transfected HEK293T cells with NgR and observed that conditioned medium from these cells contains a low but detectable level of Aβ, which is comparable to that observed in a cell expressing the FAD mutant APPsw, indicating increased β-secretase processing. The presence of NgR also increased α-secretase processing as indicated by the fact that sAPPα levels were also increased by NgR expression.

To examine the significance of the NgR/Aβ interaction on APP processing in vivo, the APPsw transgene from APPsw/PSEN-1(DeltaE9) mice was bred onto a NgR null background. Brain extracts were examined for Aβ and sAPPα levels at 3 months of age as follows. Forebrain was extracted with 0.1M formic acid, neutralized with Tris and clarified by centrifugation at 10,000×g. The levels of sAPPα were measured in the brain extracts by immunoprecipitation with anti-amino-terminal-APP 22C11 antibody (Chemicon) and by immunoblot with anti-Aβ(1′-17) 6E10 antibody (Chemicon). Compared to littermate matched control mice, the absence of NgR significantly reduces the production of both Aβ and sAPPα under physiologic conditions. These results confirmed that NgR has a role in increased Aβ formation in vivo.

### Example 5

### Fibrillogenic Aβ42 Peptide Facilitates Binding of Aβ Peptide to NgR

We examined whether the interaction between NgR and Aβ peptide has a role in aggregate formation. sNgR310 was immobilized on microtiter plates and Biotin-Aβ40 was applied along with Aβ42 peptide. We quantified bound Biotin-Aβ40 peptide using streptavidin HRP and found that increasing the concentration of Aβ42 peptide enhanced the binding of Biotin-Aβ40 peptide in a dose-dependent manner. We confirmed these data using SKNMC cells expressing human NGR1 and found that Aβ42 peptide again enhanced the binding of Biotin-Aβ40 peptide to the cells in a dose-dependent manner. We also found that anti-NgR antibodies inhibit Aβ42-peptide-mediated enhancement of Biotin-Aβ40 binding to SKNMC cells expressing human NgR1. These results indicate that interference with the NgR/Aβ peptide interaction inhibits formation of Aβ peptide aggregates.

### Example 6

### Treatment with a NgR Antagonist Reduces Aβ Plaque Deposition

To examine the role of the NgR/APP/AB interactions in vivo, sNgR310-Fc (a NgR antagonist; see International Patent Application PCT/US03/25004) was infused into APPsw/PSEN-1(DeltaE9) double transgenic mice (from Jackson Laboratories). The sNgR310-Fc protein contains the entire LRR ligand-binding of the NgR fused to the Fc portion of IgG. To administer sNgR310-Fc protein, 5-month-old mice were anesthetized with isoflurane/oxygen and a burr hole was drilled in the skull. A cannula (ALZET brain infusion kit II, Alza Scientific Products, Palo Alto, Calif.) was introduced into the right lateral ventricle at stereotaxic coordinates 0.6 mm posterior and 1.2 mm lateral to bregma and 4.0 mm deep to the pial surface. The cannula was held in place with cyanoacrylate and the catheter was attached to a subcutaneous osmotic minipump (Alzet 2ML4). The pump delivered 2.5 μl/hr for 28 days of a 1.2 mg/ml solution of sNgR310-Fc or rat IgG in PBS (control mice received rat IgG since both the NgR and the Pc moiety were of rat origin). Pumps were replaced after 28 days and connected to the same cannula. The total dose of protein infused was 2.5 mg per mouse over 56 days. At the end of this period, mice were sacrificed and brain AB levels were measured using an ELISA kit from Biosource International according to the manufacturer's instructions. Aβ deposition into amyloid plaques was assessed by anti-Aβ immunohistochemistry as follows. Aβ plaques in sagittal sections of 4% paraformaldehyde fixed brain were detected immunohistologically with anti-Aβ(1-17) 6E10 antibody after 0.1 M formic acid treatment for antigen recovery. Plaque area was quantitated using NIH Image as a percentage of cerebral cortical area for 3 sections from each animal.

In the sNgR310-Fc treated mice, the deposition of immunoreactive Aβ into plaque was significantly reduced. In addition, the total level of both Aβ(1-40) and Aβ(1-42) decreased by 50% in the brain of these mice. There was a tight correlation between Aβ levels and amyloid plaque deposition in these mice, suggesting that sNgR310-Fc alters APP/Aβ metabolism to a greater extent than Aβ aggregation. However, our data indicate that the presence of sNgR310-Fc decreases both the production of Aβ as well as its deposition in plaques. The α-secretase product, sAPPα, also was measured by immunoprecipitation and immunoblot analysis. The sAPPα levels decreased in the brains of the sNgR310-Fc treated animals to a similar extent as did the Aβ levels demonstrating that both α-secretase and β-secretase processing are inhibited by sNgR310-Fc in vivo.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-41. (canceled)

42. A method for reducing the levels of Aβ peptide in a mammal, comprising administering a therapeutically effective amount of a soluble Nogo receptor antagonist.

43. The method of claim 42, wherein the levels of Aβ peptide are elevated in association with a disease, disorder or condition.

44. The method of claim 43, wherein said disease, disorder or condition is Alzheimer's disease.

45. The method of claim 42, wherein the soluble Nogo receptor polypeptide is administered by bolus injection or chronic infusion.

46. The method of claim 45, wherein the soluble Nogo receptor polypeptide is administered directly into the central nervous system.

47. The method of claim 42, wherein the soluble Nogo receptor polypeptide is a soluble form of a mammalian NGR1.

48. The method of claim 47, wherein the soluble form of a mammalian NgR1 comprises a peptide selected from the group consisting of:
(a) amino acids 26 to 310 of human NgR1 (SEQ ID NO:3) with up to ten conservative amino acid substitutions;
(b) amino acids 26 to 344 of human NGR1 (SEQ ID NO:4) with up to ten conservative amino acid substitutions;
(c) amino acids 27 to 310 of rat NgR1 (SEQ ID NO:5) with up to ten conservative amino acid substitutions; and
(d) amino acids 27 to 344 of rat NgR1 (SEQ ID NO:6) with up to ten conservative amino acid substitutions.

49. The method of claim 48, wherein the soluble form of a mammalian NGR1 comprises a peptide selected from the group consisting of:
(a) amino acids 26 to 310 of human NGR1 (SEQ ID NO:3);
(b) amino acids 26 to 344 of human NGR1 (SEQ ID NO:4);
(c) amino acids 27 to 310 of rat NgR1 (SEQ ID NO:5); and
(d) amino acids 27 to 344 of rat NGR1 (SEQ ID NO:6).

50. The method of claim 47, wherein the soluble form of a mammalian NGR1 further comprises a fusion moiety.

51. The method of claim 42, wherein the therapeutically effective amount is from 0.001 mg/kg to 10 mg/kg of soluble Nogo receptor polypeptide.

52. A method of preventing or treating a disease, disorder or condition associated with plaques of Aβ peptide in a mammal, comprising administering a therapeutically effective amount of a soluble Nogo receptor polypeptide.

53. The method of claim 52, wherein said disease, disorder or condition is Alzheimer's Disease.

54. The method of claim 52, wherein the soluble Nogo receptor polypeptide is administered by bolus injection or chronic infusion.

55. The method of claim 54, wherein the soluble Nogo receptor polypeptide is administered directly into the central nervous system.

56. The method of claim 52, wherein the soluble Nogo receptor polypeptide comprises a soluble form of a mammalian NgR1.

57. The method of claim 56, wherein the soluble form of a mammalian NgR1 comprises a peptide selected from the group consisting of:
(a) amino acids 26 to 310 of human NGR1 (SEQ ID NO:3) with up to ten conservative amino acid substitutions;
(b) amino acids 26 to 344 of human NGR1 (SEQ ID NO:4) with up to ten conservative amino acid substitutions;
(c) amino acids 27 to 310 of rat NGR1 (SEQ ID NO:5) with up to ten conservative amino acid substitutions; and
(d) amino acids 27 to 344 of rat NGR1 (SEQ ID NO:6) with up to ten conservative amino acid substitutions.

58. The method of claim 57, wherein the soluble form of a mammalian NgR1 comprises a peptide selected from the group consisting of:
(a) amino acids 26 to 310 of human NgR1 (SEQ ID NO:3);
(b) amino acids 26 to 344 of human NgR1 (SEQ ID NO:4);
(c) amino acids 27 to 310 of rat NgR1 (SEQ ID NO:5); and
(d) amino acids 27 to 344 of rat NgR1 (SEQ ID NO:6).

59. The method of claim 56, wherein the soluble form of a mammalian NgR1 further comprises a fusion moiety.

60. The method of claim 52, wherein the therapeutically effective amount is from 0.001 mg/kg to 10 mg/kg of soluble Nogo receptor polypeptide.

61. A method for reducing the levels of Aβ peptide in a mammal, comprising administering a therapeutically effective amount of an antibody or antigen-binding fragment thereof that binds to a mammalian NGR1.


SEQUENCE LISTING (22 sequences)

SEQ ID NO: 1 - protein, 344 aa, Homo sapiens
MKRASAGGSRLLAWVLWLQAWQVAAPCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 2 - protein, 344 aa, Rattus norvegicus
MKRASSGGSRLPTWVLWLQAWRVATPCPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 3 - protein, 285 aa, Homo sapiens
PCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCA

SEQ ID NO: 4 - protein, 319 aa, Homo sapiens
PCPGACVCYNEPKVTTSCPQQGLQAVPVGIPAASQRIFLHGNRISHVPAASFRACRNLTILWLHSNVLARIDAAAFTGLALLEQLDLSDNAQLRSVDPATFHGLGRLHTLHLDRCGLQELGPGLFRGLAALQYLYLQDNALQALPDDTFRDLGNLTHLFLHGNRISSVPERAFRGLHSLDRLLLHQNRVAHVHPHAFRDLGRLMTLYLFANNLSALPTEALAPLRALQYLRLNDNPWVCDCRARPLWAWLQKFRGSSSEVPCSLPQRLAGRDLKRLAANDLQGCAVATGPYHPIWTGRATDEEPLGLPKCCQPDAADKA

SEQ ID NO: 5 - protein, 284 aa, Rattus norvegicus
CPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCA

SEQ ID NO: 6 - protein, 318 aa, Rattus norvegicus
CPGACVCYNEPKVTTSRPQQGLQAVPAGIPASSQRIFLHGNRISYVPAASFQSCRNLTILWLHSNALAGIDAAAFTGLTLLEQLDLSDNAQLRVVDPTTFRGLGHLHTLHLDRCGLQELGPGLFRGLAALQYLYLQDNNLQALPDNTFRDLGNLTHLFLHGNRIPSVPEHAFRGLHSLDRLLLHQNHVARVHPHAFRDLGRLMTLYLFANNLSMLPAEVLVPLRSLQYLRLNDNPWVCDCRARPLWAWLQKFRGSSSGVPSNLPQRLAGRDLKRLATSDLEGCAVASGPFRPFQTNQLTDEELLGLPKCCQPDAADKA

SEQ ID NO: 7 - protein, 22 aa, Artificial Sequence
  Description of Artificial Sequence Synthetic peptide
AAAFTGLTLLEQLDLSDNAQLR

SEQ ID NO: 8 - protein, 10 aa, Artificial Sequence
  Description of Artificial Sequence Synthetic peptide
LDLSDNAQLR

SEQ ID NO: 9 - protein, 10 aa, Artificial Sequence
  Description of Artificial Sequence Synthetic peptide
LDLSDDAELR

SEQ ID NO: 10 - protein, 11 aa, Artificial Sequence
  Description of Artificial Sequence Synthetic peptide
LDLASDNAQLR

SEQ ID NO: 11 - protein, 11 aa, Artificial Sequence
  Description of Artificial Sequence Synthetic peptide
LDLASDDAELR

SEQ ID NO: 12 - protein, 11 aa, Artificial Sequence
  Description of Artificial Sequence Synthetic peptide
LDALSDNAQLR

SEQ ID NO: 13 - protein, 11 aa, Artificial Sequence
  Description of Artificial Sequence Synthetic peptide
LDALSDDAELR

SEQ ID NO: 14 - protein, 11 aa, Artificial Sequence
  Description of Artificial Sequence Synthetic peptide
LDLSSDNAQLR

SEQ ID NO: 15 - protein, 11 aa, Artificial Sequence
  Description of Artificial Sequence Synthetic peptide
LDLSSDEAELR

SEQ ID NO: 16 - protein, 12 aa, Artificial Sequence
  Description of Artificial Sequence Synthetic peptide
DNAQLRVVDPTT

SEQ ID NO: 17 - protein, 6 aa, Artificial Sequence
  Description of Artificial Sequence Synthetic peptide
DNAQLR

SEQ ID NO: 18 - protein, 16 aa, Artificial Sequence
  Description of Artificial Sequence Synthetic peptide
ADLSDNAQLRVVDPTT

SEQ ID NO: 19 - protein, 16 aa, Artificial Sequence
  Description of Artificial Sequence Synthetic peptide
LALSDNAQLRVVDPTT

SEQ ID NO: 20 - protein, 16 aa, Artificial Sequence
  Description of Artificial Sequence Synthetic peptide
LDLSDNAALRVVDPTT

SEQ ID NO: 21 - protein, 16 aa, Artificial Sequence
  Description of Artificial Sequence Synthetic peptide
LDLSDNAQLHVVDPTT

SEQ ID NO: 22 - protein, 16 aa, Artificial Sequence
  Description of Artificial Sequence Synthetic peptide
LDLSDNAQLAVVDPTT